(19) United States
(12) Patent Application Publication
(10) Pub. No.: US 2005/0070761 A1
(43) Pub. Date: Mar. 31, 2005

(54) Electronic endoscope apparatus

(75) Inventor: Mitsuru Higuchi, Saitama-shi (JP)

Correspondence Address: SNIDER & ASSOCIATES, P. O. BOX 27613, WASHINGTON, DC, 20038-7613, US

(73) Assignee: Fuji Photo Optical Co., Ltd., Saitama-shi (JP)

(21) Appl. No.: 10/950,623

(22) Filed: Sep. 28, 2004

(30) Foreign Application Priority Data
Sep. 29, 2003 (JP) 2003-336718

(51) Int. Cl. A61B 1/04

(52) U.S. Cl. 600/109; 600/118

(57) ABSTRACT

An electronic endoscope apparatus has the structure that an electronic scope is detachably connected to a processor unit. This processor unit comprises image storage memory which stores an object image imaged by a CCD, a signal processing circuit for outputting this image to external equipment, a scope power-off operation switch located separately from a main-power switch are provided. Only a scope power supply unit which supplies power to the electronic scope is turned off by this scope power-off switch. At this time, when a CCD drive pulse is being outputted or when communication between with the electronic scope is being performed, the outputting of this CCD drive pulse or the communication is stopped, and thereafter,the scope power supply unit is turned off. Thereby, the electronic scope can be removed from the processor unit which is outputting an image signal.

### BACKGROUND OF THE INVENTION

The application claims the priority of Japanese Patent Applications No. 2003-336718 filed on Sep. 29, 2003 which is incorporated herein by reference.

1. Field of the Invention

The present invention relates to an electronic endoscope apparatus, and in particular, to the construction of an electronic endoscope apparatus where an imaging signal obtained by a solid state image pickup device of an electronic scope is outputted to a processor unit, and this processor unit forms a digital imaging signal for outputting the imaging signal to external digital processing equipment.

2. Description of the Related Art

An electronic endoscope apparatus is equipped with a solid-state image pickup device such as a CCD (Charge Coupled Device) in an end section of an electronic scope (electronic endoscope). This CCD images an object on the basis of the illumination of light from light equipment. It becomes possible to display an image of the object on a monitor and to record a still image etc. on a recording apparatus, by outputting the imaging signal obtained by this CCD to a processor unit, and giving various kinds of picture processing in the processor unit.

As shown also in Japanese Patent Laid-Open No. 2000-287203, this kind of electronic endoscope apparatus performs not only analog processing for outputting an object image to a normal NTSC system monitor, but also digital image processing to output the object image to various kinds of external digital equipment such as a personal computer monitor and use the image.

By the way, in an electronic endoscope apparatus, since an image (moving image and still image) was displayed on an NTSC (PAL) system monitor, the signal processing was performed on the basis of the display pixel count (horizontal pixel count and vertical pixel count) of this NTSC system also in the above-mentioned digital image processing. But, in recent years, since CCDs which are solid state image pickup devices have been made to be in high pixel counts and high resolutions, it is proposed to form digital images where image information obtained by the CCDs with high pixel counts is employed efficiently. That is, as display standards for personal computers etc., there are standards such as VGA (Video Graphics Array) , XGA (Extended Graphics Array), and SXGA (Super XGA) whose display pixel counts are different. When forming an image signal corresponding to one of these standards, it becomes possible to use a high-resolution image in external digital equipment.

Nevertheless, since the size of one high-resolution image is large (an amount of data per image is large), digital image processing time (transmission time) becoming long. Hence, since the processing of outputting a digital image signal to external equipment is not completed in spite of endoscopy being ended, there arises a problem that the electronic scope cannot be removed from the processor unit. That is, although washing and disinfection of the electronic scope must be performed and connection of another electronic scope is necessary in preparation for the next examination after endoscopy is ended, the next operation cannot be performed smoothly when the output processing of the digital image signal to the external equipment is not completed.

The present invention is made in view of the above-mentioned problem, and aims at providing an electronic endoscope apparatus where an electronic scope can be removed from a processor unit even when the outputting of a digital image signal to external equipment is not completed.

### SUMMARY OF THE INVENTION

In order to attain the above-mentioned object, the present invention is characterized in that, in an electronic endoscope apparatus constructed so that an electronic scope in which a solid state image pickup device is mounted may be detachably connected to a processor unit and power may be supplied to the electronic scope from this processor unit, the processor unit comprises image storage memory which stores an object image imaged by the electronic scope, a signal processing circuit for outputting an image stored in this image storage memory to external equipment, scope power-off operation-means located separately from a processor main-power switch, and a control circuit which turns off (cuts off) power supply to the electronic scope on the basis of the OFF operation by this scope power-off operation means.

It is preferable that, when a CCD drive pulse is being outputted to the electronic scope from the processor unit, the above-mentioned control circuit will turn off the power supply to the electronic scope after the outputting of the CCD drive pulse is stopped.

In addition, it is preferable that, when the processor unit is communicating with the electronic scope, the control circuit will turn off the power supply to the electronic scope after the communication between the processor unit and electronic scope is stopped.

According to the above-mentioned structure, when the scope power-off switch located at, for example, a control panel of the processor unit is pushed, the power supply to the electronic scope is stopped by the control circuit. In addition, when the CCD drive pulse is being supplied to the electronic scope, or when communication is being performed between with the electronic scope, the power supply to the electronic scope will be turned off after these operations are stopped. At this time, the power of the processor unit is not turned off, but the processing of outputting the digital image stored in the image storage memory to the external equipment is performed continuously.

According to such electronic endoscope apparatus, the electronic scope can be removed from the processor unit regardless of whether the output processing of the digital image to the external equipment is completed. Hence, it becomes possible to smoothly perform the washing of the electronic scope after endoscopy is ended, the connection of another electronic scope for the following examination, and the like.

In addition, even when the CCD drive pulse is being outputted or communicating between with the electronic scope .is being performed, scope power can be disconnected safely. Hence, it becomes possible to prevent amalfunction or a failure of the apparatus, mixing of noise, etc.

### BRIEF DESCRIPTION OF THE DRAWINGS

FIG. 1 is a circuit block diagram showing the structure of an electronic endoscope apparatus according to an embodiment of the present invention; and

FIG. 2 is a schematic diagram showing the entire structure of the electronic endoscope apparatus according to the embodiment.

### DETAILED DESCRIPTION OF THE PREFERRED EMBODIMENTS

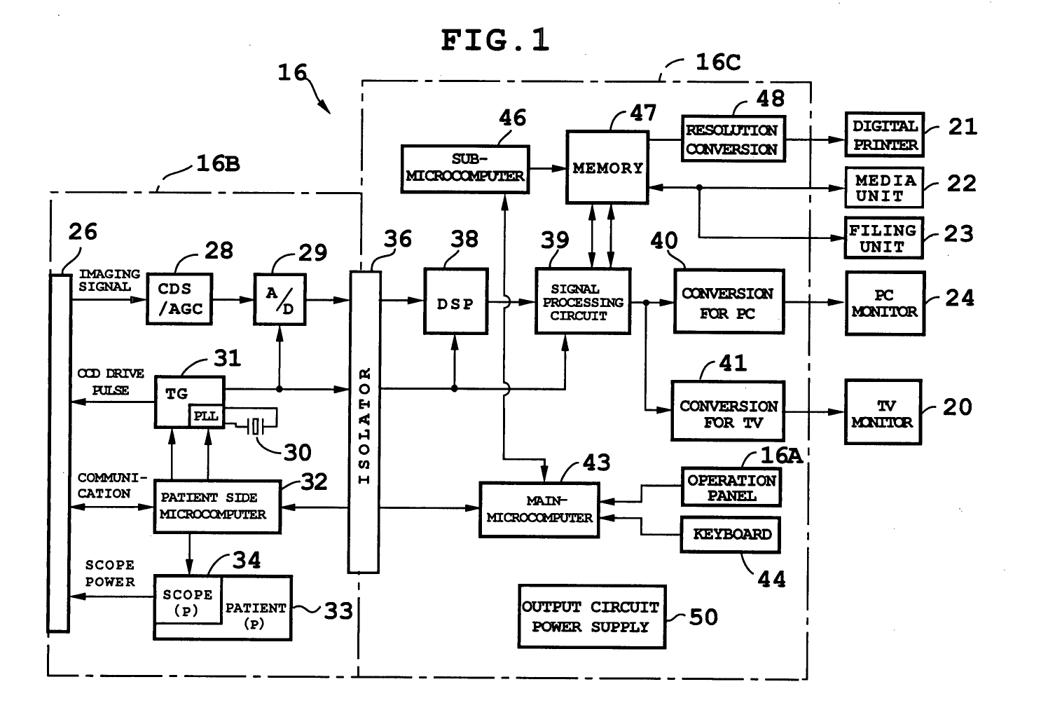

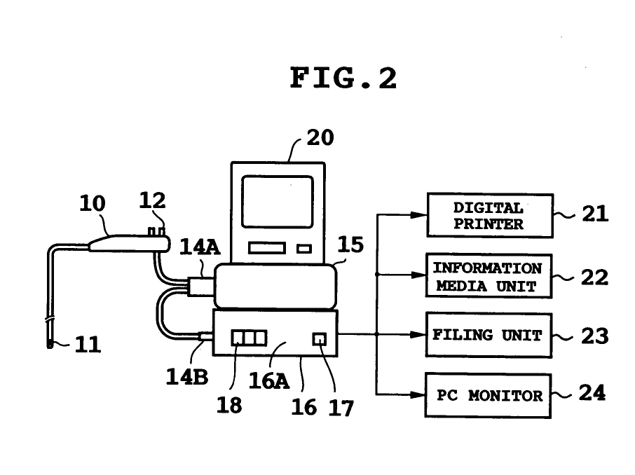

FIGS. 1 and 2 show the structure of an electronic endoscope apparatus according to an embodiment, and the entire structure will be explained first. As shown in FIG. 2, a CCD 11 which is a solid-state image pickup device is provided in an end section of an electronic scope (electronic endoscope) 10. As this CCD 11, CCDs with various kinds of pixel counts are mounted. In an operation unit of this electronic scope 10, control switches such as a freeze/record button 12 are located. This electronic scope 10 is connected not only to light equipment 15 through a light guide connector 14A, but also to a processor unit 16 through a signal/power supply line connector 14B. The light of the above-mentioned light equipment 15 is supplied to the end section through a light guide located in the electronic scope 10. An object is imaged by the above-mentioned CCD 11 owing to illumination light emitted from this end section.

In the front control (operation) panel 16A of the processor unit 16, a scope power-off switch (operation button) 18 is located as well as a main power switch (operation button) 17. In addition, although not shown, a keyboard is also connected in the processor unit 16, and hence, the scope power-off switch may be also assigned to a predetermined key in this keyboard. An NTSC system TV monitor 20, a digital printer 21, an information (recording) media unit 22, a filing unit 23, a personal computer (PC) monitor 24, and the like are connected to this processor unit 16.

FIG. 1 shows the detailed structure in the processor unit 16. This processor unit 16 has a patient circuit 16B which performs a predetermined picture processing, and an output circuit 16C which forms a signal corresponding to various kinds of output forms. The signal/power line connector 14B in the side of the above-mentioned electronic scope 10 is connected to the signal/power line connector 26. In the above-mentioned patient circuit 16B, when explaining from the upper part of the diagram, a CDS/AGC (correlation double sampling/automatic gain control) circuit 28 which samples and amplifies an imaging signal inputted from the CCD 11, an A/D converter 29, a crystal oscillator 30, a timing generator (TG) 31 which outputs a CCD drive pulse and a synchronizing signal to the electronic scope 10 and forms other timing signals, and a patient side microcomputer 32 which communicates with the electronic scope 10 and controls the patient circuit 16B are provided.

Then, a patient power supply unit (P) 33, which supplies power of this patient circuit 16B, and a scope power supply unit 34, which supplies power to the electronic scope 10 through the signal/power line connector 26 (P), are provided. The switching of the power supply in this scope power supply unit 34 is controlled by the patient side microcomputer 32.

An output circuit 16C is connected to the patient circuit 16B through an isolator 36 maintaining an electric isolation state. In this output circuit 16C, a DSP (Digital Signal Processor) 38 and a signal processing circuit 39 which give various kinds of image processing to the picture signal which is supplied from the A/D converter 29 and is digitized, a resolution conversion circuit 40 for PC which converts an output of this signal processing circuit 39 into predetermined resolution (for example, image sizes such as VGA and XGA) for displaying the output on the personal computer monitor 24, and a resolution conversion circuit 41 for TV which converts the output of this signal processing circuit 39 into an analog signal (Y/C signal etc.) in resolution (image size) for displaying the output on the NTSC (PAL) system TV monitor 20 are provided. These resolution conversion circuits 40 and 41 have each image memory which stores images at the time of freeze.

In addition, a main microcomputer 43 which totally controls the circuits in the processor unit 16 is provided. Control signals of the switches 17, 18, and the like which are located at the operation panel 16A, and a key operation signal from the keyboard 44 are supplied to this main microcomputer 43. This microcomputer 43 transmits the control signals to the patient side microcomputer 32 when the scope power-off switch 18 is pushed. Furthermore, a sub-microcomputer 46 which controls external digital equipment etc. (control of writing, reading, etc. of image data to the below-mentioned memory 47), image storage memory 47 which can store at least one set of examination data (for example, nearly 100 images) so as to output examination images to the external equipment, and a resolution conversion circuit 48 which forms a digital image signal corresponding to standards such as VGA, XGA, and SXGA so as to output the examination images to the digital printer 21 are provided. As this resolution conversion circuit 48 or the resolution conversion circuit 40 for PC, there is, for example, a DVI (Digital Video Image) circuit which performs the parallel-to-serial conversion of the image signal which is given resolution conversion and outputs this serial signal as a differential signal. In addition, an output circuit power supply unit (P) 50 is provided in this output circuit 16C.

This embodiment has the above construction, and when the main power supply switch 17 of the control panel 16A is pushed, power is supplied from the power supply units 50, 33, and 34 to each circuit, and image pickup by the CCD 11 at the end of the electronic scope 10 is started. A signal outputted from this CCD 11 is sampled by the CDS/AGC circuit 28, is converted into a digital signal by the A/D converter 29, and thereafter, is given various kinds of image processing by the DSP 38 and signal processing circuit 39. Then, this picture signal is supplied through the resolution conversion circuit 40 for PC to the PC monitor 24, or through the resolution conversion circuit 41 for TV to the TV monitor 20, and the object image is displayed on each monitor. In addition, when the freeze switch of the freeze/record button 12 of the electronic scope 10 which is a first step is pushed, the still image contained in the image memory in each of the resolution conversion circuits 40 and 41 (frame memory etc.) is displayed on the TV monitor 20 or PC monitor 24. In addition, this image data at the time of freeze is supplied also to the image storage memory 47.

When the recording switch of the freeze/record button 12 which is a second step is pushed with looking at the display image of the TV monitor 20, the writing state of the still image currently supplied in the image storage memory 47 is maintained. Then, when being read by the read out control of the sub-microcomputer 46, this still picture data is outputted to the information media unit 22 or the filing unit 23. In addition, a still image in the image storage memory 47 is converted by the resolution conversion circuit 48 into one of image sizes (resolution) such as VGA and XGA, and this image is outputted to the digital printer 21 and the like.

In such a recording operation, the image storage memory 47 has the capacity of being able to store at least one examination set of image data. Hence, observation and recording of the next image are possible without waiting the output completion of image data every recording operation to the external equipment (21 to 23). Thereby, the delay of the recording operation caused by the increase of data volume per image in connection with recent tendency of CCDs to high-pixel counts is canceled. Nevertheless, on the contrary, there arises the situation that, even after the endoscopy for a patient is ended, the recording operation to the external equipment 21 to 23 is not completed.

Then, in the present invention, the supply of scope power can be controlled. Hence, when the scope power-off switch 18 of the control panel 16A is pushed, the main microcomputer 43 outputs a command of scope power-off to the patient side microcomputer 32. Then, only the scope power supply unit 34 is turned off by the control of this patient side microcomputer 32. Namely, when the CCD drive pulse is being outputted from the timing generator 31, the outputting of this CCD drive pulse is stopped (cut off) first. Next, when the patient side microcomputer 32 is communicating with the electronic scope 10, this communication is stopped (cut off), and thereafter, the scope power supply unit 34 is turned off.

At this time, since the patient power supply unit 33 and output circuit power supply unit 50 are not turned off, the outputting (writing) of the image data to the external equipment 21 to 23 is performed continuously. Accordingly, since the electronic scope 10 can be removed from the processor unit 16 before the recording operation is completed, it becomes possible to quickly perform subsequent operations such as washing of the electronic scope 10. In addition, in this embodiment, the outputting of the CCD drive pulse and the communication between with the electronic scopes 10 are turned off before the scope power supply unit 34 is turned off as mentioned above. Hence, there are advantages that it is possible to prevent amalfunction and a failure of the apparatus by these operations, and that it is also possible to prevent the mixing of noise.

The case that a still image is outputted to the external equipment 21 to 23 is explained in the above-mentioned embodiment. But, the present invention can be also applied to the case that moving image memory is provided as the image storage memory and the moving images are outputted to external equipment for moving images. In addition, although the switch 18 of the control panel 16A is made the scope power-off operation means, other operation members such as a keyboard may be also used as this operation means, or it may be also located in the side of the electronic scope 10.

1. An electronic endoscope apparatus, comprising:
an electronic scope in which a solid state image pickup device is mounted; and
a processor unit detachably connecting this electronic scope and supplying power of this electronic scope, wherein the processor unit comprises:
image storage memory which stores an object image imaged by the electronic scope;
a signal processing circuit for outputting an image stored in this image storage memory to external equipment;
scope power-off operation means located separately from a processor main-power switch; and
a control circuit which turns off power supply to the electronic scope on the basis of OFF operation by the scope power-off operation means.

2. The electronic endoscope apparatus according to claim 1, wherein, when a CCD drive pulse is being outputted to the electronic scope from the processor unit, the control circuit turns off power supply to the electronic scope after stopping outputting of the CCD drive pulse.

3. The electronic endoscope apparatus according to claim 1, wherein, when the processor unit is communicating with the electronic scope, the control circuit turns off power supply to the electronic scope after the communication between the processor unit and the electronic scope is stopped.
